(19) United States
(12) Patent Application Publication
(10) Pub. No.: US 2022/0008610 A1
(43) Pub. Date: Jan. 13, 2022

(54) HEMOSTATIC SPONGY MATERIAL OR TISSUE SEALANT AND METHOD THEREOF

(71) Applicant: Xiamen University, Xiamen (CN)

(72) Inventors: Jun CHEN, Xiamen (CN); Jingwei CHEN, Xiamen (CN); Dexiang WANG, Xiamen (CN); Shaoxiong DING, Xiamen (CN); Jing ZHAO, Xiamen (CN); Huilong OU, Xiamen (CN); Guangyuan XIA, Xiamen (CN)

(21) Appl. No.: 17/486,866

(22) Filed: Sep. 27, 2021

Related U.S. Application Data
(63) Continuation of application No. PCT/CN2019/087551, filed on May 20, 2019.

(30) Foreign Application Priority Data
Mar. 27, 2019 (CN) 201910239309.9

(51) Int. Cl. A61L 24/00

(52) U.S. Cl. CPC A61L 24/0036; A61L 2300/418; A61L 2400/04; A61L 24/0005

(57) ABSTRACT

A hemostatic material or a tissue sealant, wherein a main component of the hemostatic material or the tissue sealant or part of the hemostatic material or the tissue sealant is a sponge grown in sea water or fresh water.

### RELATED APPLICATIONS

This application a continuation of International patent application of PCT/CN2019/087551, filed on May 20, 2019, which claims priority to Chinese patent application 201910239309.9, filed on Mar. 27, 2019. International patent application of PCT/CN2019/087551 and Chinese patent application 201910239309.9 are incorporated herein by reference.

### FIELD OF THE DISCLOSURE

The present disclosure relates to a hemostatic material or a tissue sealant and a method thereof, and in particular relates to a natural spongy material.

### BACKGROUND OF THE DISCLOSURE

In an emergency or large-scale disaster situation, the surviving victims are often killed by subsequent uncontrollable bleeding. According to statistics, between 2011 and 2013, about 600,000 people died in traffic accidents in China each year, of which 85% were caused by massive bleeding during the initial stage of trauma. If bleeding can be urgently treated and controlled within 30 minutes, the survival rate of the injured will be able to increase by more than 40%. In war, emergency and effective hemostatic treatment is one of the most effective measures to reduce the casualties of soldiers. Hemostatic drugs are a strategic area that the military needs to develop. Additionally, in daily life, small scale wound bleeding caused by most normal accidents is one of the inevitable problems that people frequently experience, and the key to the success of an operation lies in whether the patient's bleeding can be controlled. At the same time, the large group of people with hemophilia have blood clotting problems, and a lot of drugs are required to supplement normal life. It can be said that in the race between life and death, our body is constantly experiencing the challenge of how to quickly repair after bleeding. Therefore, the development of high-efficiency, hemostatic drugs has always been the focus of attention of scientific researchers in the medical field and a problem that needs continuous breakthroughs.

After the bleeding caused by the wound occurs, the body's own repair mechanism combines the joint action of blood vessels, platelets, fibrinolytic balance system, and blood coagulation system. Blood vessel walls slow down the blood flow through contraction, and the platelets adhere to the surface of foreign bodies, deform, and release a coagulation factor to activate thromboplastin in the blood. The generated thromboplastin acts on dissolved fibrinogen in the blood, turns the dissolved fibrinogen into solid fibrin to connect into a network structure to adsorb red blood cells, and finally forms a thrombus on the surface of the wound under the combined action of the fibrin network and platelets to inhibit bleeding.

For the above-mentioned different stages of coagulation process and mechanism, various types of coagulation drugs have been developed, which can basically be divided into coagulation factors, adhesions, and procoagulants. Coagulation factors, such as the most normal quick-acting hemostatic powder, are mainly composed of inorganic particles such as zeolite and kaolin. The principle is to slow the bleeding rate by absorbing large amounts of water in the plasma of the wound through its van der Waals force, concentrate the clotting factors, and accelerate the formation of blood clots by exothermic reaction. This type of product is one of the earliest hemostatic drugs, which also shined in the Iraq War, but its shortcomings cannot be ignored. First of all, local high temperature will burn the tissue wound, which is not conducive to the subsequent wound repair. At the same time, small particles of inorganic matter can easily enter the blood and cause pulmonary thrombosis. Current mainstream hemostatic products on the market are collagen and chitosan polysaccharides, which are adhesive products. Collagen itself has very strong water absorption, and also has good affinity and adsorption to platelets. Collagen can quickly concentrate and activate platelets at the wound and promote the formation of blood clots. However, collagen can promote the growth of bacteria, leading to frequent contamination of wounds and infections, and adhesion of collagen is poor and easily falls off. Therefore, the development direction of collagen is mostly surgical suture products. Chitosan itself has excellent antibacterial properties and histocompatibility. The modified chitosan can adsorb negatively charged red blood cells through the positive charge of the amino group, so that the red blood cells can aggregate and adhere. The disadvantage of chitosan is that chitosan has limited hemostatic effect and cannot address extensive bleeding wounds. However, chitosan is still a very ideal hemostatic base material, and chitosan is the focus of the current research on multi-material composite hemostatic agents. There is also a special type of hemostatic drugs that does not have any endogenous hemostatic function, but uses exogenous substances to quickly cross-link and polymerize at the wound to achieve the purpose of rapid adhesion of the wound and sealing of the injured tissue. The main products are synthetic polymer materials such as α-cyanoacrylate and polyethylene glycol. Although the wound can be closed quickly, harmful substances will be produced in the later degradation process to cause inflammation and necrosis of the tissue, so the application situations are more unique.

The above hemostatic materials also have a major common shortcoming. For patients with blood coagulation disorders, the above-mentioned drugs are often incapable of stopping bleeding from their wounds. Therefore, it is very necessary to develop hemostatic drugs that supplement coagulation factors. These kinds of procoagulant drugs carry high concentrations of fibrinogen, thrombin, and other procoagulant factors to complete the three stages of coagulation, but the cost is often very expensive, and at the same time, procoagulant drugs need a normal coagulation stage, so procoagulant drugs need a certain coagulation time. Compared with the hemostatic material, the effect of promoting the coagulation rate is not obvious, and procoagulant drugs are not suitable for large-area bleeding. In particular, soluble procoagulant hemostatic drugs can interfere with the balance of the fibrinolytic system, easily cause thrombosis in the body, and bury unnecessary safety hazards. All of the above limit the use of such drugs.

### BRIEF SUMMARY OF THE DISCLOSURE

An objective of the present disclosure is to provide a new hemostatic material, which can simultaneously cover the advantages of multiple types of hemostatic drugs, can be used in special conditions such as platelet deficiency and hemophilia lacking clotting factors, and can help patients to stop bleeding through wounds and can reduce drug cost.

The technical solutions adopted by the present disclosure to solve the foregoing technical problems are: a hemostatic material or tissue sealant, main component or part component is a sponge that grows in the ocean, sea water, or fresh water. In particular, fleshy components of the sponge are removed.

Among the sponges, one of the oldest organisms in the ocean, its unique organic substance, spongy, is a complex collagen-like macromolecular substance with a triple helix structure, and the spongy itself has a complex three-dimensional mesh skeleton structure that can be used to anchor platelets to fix blood clots.

A method for preparing a hemostatic spongy material (or a tissue sealant), comprising the following steps:

(1) taking an adult sponge (e.g., a fresh adult sponge) and washing away fleshy components on a surface of the adult sponge (e.g., by physical methods), keeping a skeleton of the adult sponge;

(2) immersing the skeleton of step (1) in a hydrochloric acid (HCl) solution with a concentration of 0.7 to 0.8 mol/L for 2 to 3 days, taking the skeleton out, and washing the skeleton several times with clean water;

(3) immersing the skeleton of step (2) in an NaOH solution with a concentration of 0.1 to 0.2 mol/L for 2 to 3 days, taking the skeleton out, and immersing the skeleton in clean water for 3 to 4 days;

(4) adding the skeleton of step (3) to a Tris-HCl buffer solution, stirring and pulverizing into a homogenous suspension, wherein a concentration of the Tris-HCl buffer solution is 0.1M and a pH is 7.8 at 37° C.; adding 10% trypsin to the homogenous suspension, shaking for enzymolysis for 2 to 3 days to obtain a product;

(5) filtering and separating the product of step (4) to obtain a separated precipitate, immersing and washing the separated precipitate with clean water 2 to 4 times, and drying to obtain spongy precipitate, wherein the spongy precipitate is an insoluble, large branch fiber spongin B;

(6) at least one of:
breaking the spongy precipitate of step (5) into small particles with a freezing grinder, and sieving to obtain spongy powders SFM; or
using a hydrogen peroxide method to degrade the spongy precipitate of step (5) into soluble spongy materials SR.

In a preferred embodiment of the present disclosure, the adult sponge is normally called bath sponge, most of which is species from the order Dictyoceratida (class of Demospongiae).

In a preferred embodiment of the present disclosure, in step (1), the adult sponge is decayed to decompose an epidermis and the fleshy components, or the adult sponge is put in sea water and vigorously washed to remove the fleshy components on the surface of the adult sponge.

In a preferred embodiment of the present disclosure, in step (2), the HCl solution has a concentration of 0.8 mol/L, and an immersing time is 2 days.

In a preferred embodiment of the present disclosure, in step (3), the NaOH solution has a concentration of 0.1 mol/L.

In a preferred embodiment of the present disclosure, in step (6), a 200-mesh sieve is used to obtain the spongy powders SFM.

In a preferred embodiment of the present disclosure, the hemostatic spongy material comprises spongy powders SFM and soluble spongy materials SR, and the hemostatic spongy material has a scattered pore structure.

Compared with the existing techniques, the present disclosure has the following advantages.

1. The hemostatic material has a scattered pore structure, so that the material itself has water absorption and air permeability comparable to medical cotton. Strong water absorption can help increase the concentration of coagulation factors near the wound, and excellent air permeability helps the normal metabolism of the tissues near the wound and facilitates subsequent repairs.

2. The hemostatic material combines the advantages of collagen and chitosan at the same time and shows excellent enrichment and adsorption capacity for red blood cells and platelets.

3. The hemostatic material has good biocompatibility. It is observed under the electron microscope that platelets can quickly activate and differentiate when contacted with the hemostatic material, and the red blood cells actively undergo benign deformation after contact with the hemostatic material and stick out the artificial foot for attachment.

4. The hemostatic material has quite excellent hemostatic properties has special hemostatic capabilities that collagen and other normal hemostatic drugs do not possess, and can effectively act in special situations where blood cannot be coagulated due to coagulation factor defects. Without the participation of fibrinogen and platelets, the blood can still be coagulated normally.

5. The hemostatic material is derived from marine organisms and is a pure natural biological material. Being alienated from humans, there is no risk of infectious diseases. It has the advantages of low sensitivity, safety, and low cost.

### BRIEF DESCRIPTION OF THE DRAWINGS

FIGS. 1A-1D illustrate morphological variations of red blood cells in a red blood cell adsorption performance detection, wherein FIG. 1A illustrates original sheep red blood cells, FIG. 1B illustrates red blood cells of a blank group disposed on a silicon wafer, FIG. 1C illustrates red blood cells of a spongy powders SFM group, and FIG. 1D illustrates red blood cells of a soluble spongy materials SR coating group.

FIGS. 2A-2D illustrate the adsorption of red blood cells to various materials in the red blood cell adsorption capacity detection, wherein FIG. 2A illustrates the blank group, FIG. 2B illustrates the soluble spongy materials SR coating group, FIG. 2C illustrates an original shape of the spongy powders SFM, and FIG. 2D illustrates the spongy powders SFM accumulated with the red blood cells.

FIGS. 3A-3D illustrate the aggregation of platelets to various materials, wherein FIG. 3A illustrates original platelet shape, FIG. 3B illustrates the blank group, FIG. 3C illustrates the soluble spongy materials SR coating group, and FIG. 3D illustrates the spongy powders SFM group.

FIG. 4 illustrates coagulation characteristics of platelet-poor blood.

FIG. 5 illustrates the microscopic structure of the platelet-poor blood coagulated clot using SFM material.

FIG. 6 illustrates coagulation characteristics of sheep fibrinogen removal blood.

FIG. 7 illustrates the microscopic structure of the sheep fibrinogen removal blood coagulated clot using the spongy powders SFM.

FIG. 8 illustrates the microscopic structure of the sheep fibrinogen removal blood coagulated blood clot using the soluble spongy materials SR.

### DETAILED DESCRIPTION OF THE EMBODIMENTS

### Embodiment 1

In this embodiment, a method for preparing a hemostatic spongy material, comprises:

(1) Fresh adult Spongia officinalis sponges (i.e., Dictyoceratida sponge, S. officinalis sponges) are usually directly decayed to decompose epidermis and fleshy components of the Spongia officinalis sponges or placed in seawater and vigorously washed to remove the fleshy components on surfaces of the Spongia officinalis sponges using physical methods to obtain skeletons of the Spongia officinalis sponges. The skeletons of the S. officinalis sponges are broken into small pieces to facilitate subsequent steps.

(2) Hydrochloric acid (HCL) with a concentration of 0.8 mol/L is obtained, and the small pieces of the skeletons of the S. officinalis sponges are immersed for 2 days to remove residual surface impurities, calcium-containing complexes, and some acid-soluble proteins.

(3) Insoluble spongy skeletons are washed by clean water several times and are then immersed with 0.1 mol/L NaOH, and some residual alkali-soluble proteins and some residual alkali-soluble impurities of the insoluble spongy skeletons are cleaned.

(4) The insoluble spongy skeletons are immersed in clean water 3 times to remove residual sodium hydroxide solution, and a Tris-Hcl buffer (0.1 M (mol/L), pH 7.8, 37° C.) is added, stirred, and pulverized into a homogenous suspension. 10% trypsin is then added, shaken, and digested for 2 days.

(5) Precipitates and supernatants are separated by filtration. The supernatants are enzyme soluble intercellular linear fibers, namely spongin A and other enzyme-degradable impurities, and the precipitates are insoluble large branched fibers, namely spongin B. The spongin in the S. officinalis sponge are mainly insoluble spongin B, which comprises a more complex three-dimensional structure.

(6) The spongin B are taken and immersed in clean water 3 times and are dried.

\textcircled{1} The spongin B are broken into small particles by a freezing grinder and are screened by a 200-mesh sieve to obtain spongy powders SFM.

\textcircled{2} The spongin B are degraded into soluble spongy materials SR using a hydrogen peroxide method.

I. Hemostatic Performance Test (Blood Clotting Time and Water Absorption Rate In Vitro)

\textcircled{1} Blood Clotting Time In Vitro

Experimental method: the blood clotting time is analyzed using a glass test tube method.

Fresh chicken blood is taken by a sodium citrate blood collection tube, 1 mL of plasma is collected in a glass tube and pre-heated in a water bath pot at 41° C., 2.8 mg/mL calcium chloride solution and materials of each of the experimental groups are then added, and a whole blood clotting time is recorded. In each experimental group, the process is repeated 3 times to take an average value. The materials of the experimental groups are the soluble spongy materials SR and the insoluble spongy powder SFM, and control groups are blank, Yunnan Baiyao (purchased in the market), and collagen. Experimental results are shown in the following table.

TABLE 1
The blood clotting time of each of the experimental material groups

| Groups | 0.5 mg/mL | | 1 mg/mL | | 2 mg/mL | |
|---|---|---|---|---|---|---|
| Blank | 3 minutes 42 seconds | | | | | |
| Yunnan | 2 | minutes | 2 | minutes | 5 | minutes |
| Baiyao | 31 | seconds | 53 | seconds | 50 | seconds |
| Collagen | 3 | minutes | 2 | minutes | 4 | minutes |
| | 37 | seconds | 50 | seconds | 22 | seconds |
| SFM | 2 | minutes | 1 | minutes | 1 | minutes |
| | 24 | seconds | 58 | seconds | 36 | seconds |
| SR | 3 | minutes | 3 | minutes | 4 | minutes |
| | 41 | seconds | 14 | seconds | 28 | seconds |

It can be seen from Table 1 that a main hemostatic mechanism of the hemostatic material collagen is to increase a local blood-clotting factor concentration and to adsorb platelets due to water absorption performance, so effects in a blood-clotting experiment in vitro is not obvious. Components of Panax Notoginseng of Yunnan Baiyao promotes hemostasis, however, it is the same as the soluble spongy materials SR, the collagen, and other materials soluble in the blood, when a concentration increases, it will generate an opposite effect. It is speculated that an internal balance system of blood is broken down, and a function and a vitality of blood clotting factors and thrombin are interfered with due to a high concentration of external substances. However, the spongy powders SFM are insoluble in blood, and when the amount of the spongy powders SFM used increases, its clotting time is gradually shortened, which has a significant effect compared with the blank group.

\textcircled{2} Water Absorption Rate

Experimental method: a simulated body fluid (SBF) water absorption rate test method normally used in porous hemostatic materials is used. A SBF solution is prepared and poured into a dry petri dish. A sample is baked in an oven at 60° C. until to obtain a constant weight, about 0.5 g of the sample is weighed, an initial weight W0 is recorded, the sample is added into a petri dish, immersed for 30 minutes, and taken out, and a final weight Wt is recorded. In each sample group, the process is repeated 3 times to take an average value. It is considered that the soluble spongy materials SR are soluble in water, experimental groups only use the spongy powders SFM to function as raw materials, and cotton wool (i.e., medical cotton) is used as a control group. The water absorption rate is calculated according to the following formula:

Saturated water absorption=(Wt−W0)/W0×100%

Experimental results are shown in the following table.

TABLE 2
Water absorption rate of hemostatic materials

| | Groups | | |
|---|---|---|---|
| | medical cotton | particle spongy | Flocculent spongy |
| Water absorption | 1768.6 ± 82.7 | 994.3 ± 84.4 | 4050.3 ± 242.6 |
| rate (%) | | | |

Table 2 shows the water absorption characteristics of the spongy powders SFM. The spongy skeletons as a whole comprise rich microporous structures and have better water absorption performance compared with conventional materials on the market. After the spongy skeletons are ground to powders and are sieved, large particles of the spongy powders SFM that have not been sieved are mixed into a structure shaped as floc. Its water absorption performance is 2.5 times that of the medical cotton also shaped as floc. However, some macroscopic, microporous structures of the spongy powders SFM ground into small particles are destroyed, resulting in its water absorption capacity being reduced and still reaching half of the water absorption performance of the medical cotton. Therefore, pore structures of the spongy powders SFM are helpful for water molecule penetration. As a hemostatic powder, the spongy powders SFM can excellently absorb water from a wound, which is beneficial to increase a local blood-clotting factor concentration and accelerate blood clotting at the wound.

II. Detection of Red Blood Cell Adsorption Performance and Platelet Aggregation

\textcircled{2} Red Blood Cell Adsorption Performance

Experimental materials: fresh sheep blood, the spongy powders SFM, the soluble spongy materials SR

Experimental Method:

1. Red blood cells are acquired: the fresh sheep blood is taken and centrifuged at 1000 r/15 min (1000 revolutions/15 minutes), precipitates are obtained and washed with a phosphate buffer solution (PBS), and the process is repeated 3 times. A pure red blood cell PBS solution is obtained from sheep blood.

2. Preparation of smear:

\textcircled{1} The soluble spongy materials SR are configured into obtain a solution, the solution is dropped on a silicon wafer and is dried to form a surface thin film, and a soluble spongy materials SR group is obtained.

\textcircled{2} The spongy powders SFM are evenly scattered on the silicon wafer soaked in egg white gel and dried to be fixed, and a spongy powders SFM group is obtained.

\textcircled{3} A blank silicon wafer is cleaned by alcohol and dried.

3. The silicon wafer in an experimental group is immersed in the red blood cell PBS solution and left to stand for 30 minutes at 37° C.

4. The silicon wafer is taken, gently washed by PBS 3 times, and put into 2.5% glutaraldehyde for fixation overnight.

5. 50%, 60%, 70%, 80%, 95%, and 100% ethanol are serially added for gradient dehydration, 15 minutes each time.

6. Gold is sprayed, and red blood cell adsorption is observed by scanning electron microscope.

Experimental Results:

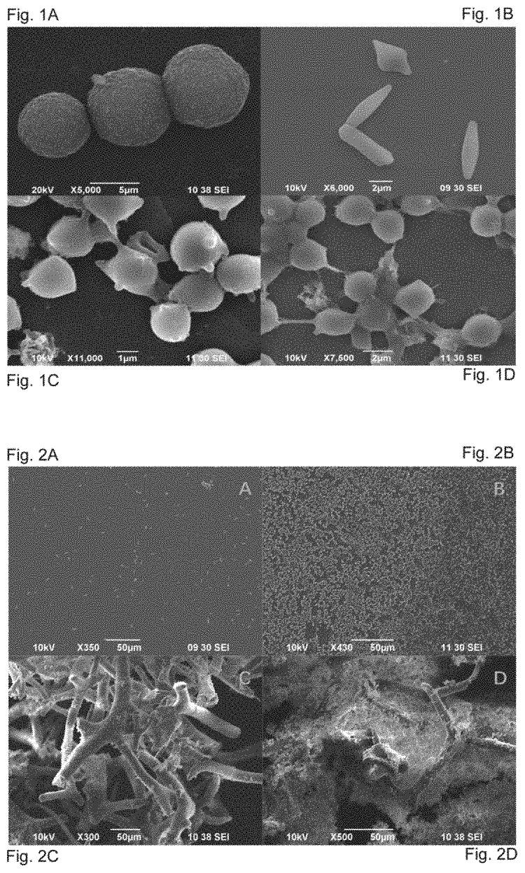

Referring to FIGS. 1A-1D, the red blood cells contact with the blank silicon wafer for 30 minutes, and the red blood cells are converted from spherical shapes into irregular granular deformations, which are a stress reaction of the red blood cells in a bad environment. Compared with the blank group, the red blood cells basically maintain healthy and complete red blood cell morphologies in the soluble spongy materials SR group and the spongy powders SFM group. At the same time, when exposed to foreign substances, it is observed that the red blood cells begin to give hemostatic feedback, form benign deforms, and extend pseudopod to be actively adsorbed on surfaces of the soluble spongy materials SR (e.g., a coating of the soluble spongy materials SR) and the spongy powders SFM as the external sources, which can enhance the adsorption performance and help stabilize the blood clot on a wound surface. Analysis shows that the soluble spongy materials SR and the spongy powders SFM have good biocompatibility to the red blood cells.

Referring to FIGS. 2A-2D, compared with the stressed red blood cells irregularly scattered on the blank silicon plate, the surface of the silicon plate with the coating of the soluble spongy materials SR is enriched with a large number of the red blood cells, and the large number of the red blood cells are still firmly adhered to the surface of the silicon plate after repeated washing with PBS due to the benign induction of pseudopodia. FIGS. 2C and 2D illustrate changes before and after the spongy powders SFM are exposed to the red blood cells. It is observed that a large area of the red blood cells is adsorbed on the surface of the spongy powders SFM. At the same time, free red blood cells aggregate around the anchored red blood cells through an intercellular macromolecular bridging force. The complicated, branched, three-dimensional structure of the spongy powders SFM also provides stable support for an aggregation of the red blood cells at multi-angles to ultimately promote a formation of the complete blood clot.

Analysis shows that the spongy materials have good biocompatibility to the red blood cells. The spongy materials can not only adsorb the red blood cells by themselves, but also induce the red blood cells to differentiate the pseudopodia for absorption. At the same time, the complex, branched structure of the insoluble spongy powders SFM can provide solid support for the aggregation of the red blood cells. It is speculated that the spongy materials can replace fibrin in the wound in practical applications, a step at which the fibrin is generated by thrombin and fibrinogen, etc. is skipped, the red blood cells are directly and quickly adsorbed to aggregate into clumps, and the spongy materials have excellent hemostasis value.

\textcircled{2} Platelet Aggregation

Experimental materials: platelet-rich plasma, the spongy powders SFM, and the soluble spongy materials SR

Experimental Method:

1. Preparation of a platelet PBS solution: the platelet-rich plasma is taken and centrifuged at 3500 r/15 min (3500 revolutions/15 minutes), the precipitate is taken and washed by PBS, and the process is repeated 3 times to obtain a platelet PBS solution.

2. Preparation of smear:

\textcircled{1} The soluble spongy materials SR are configured to a solution, and the solution is dropped on a silicon wafer and dried to form a surface thin film.

\textcircled{2} The spongy powders SFM are evenly scattered on the silicon wafer soaked in egg white and dried to be fixed.

\textcircled{3} The blank silicon wafer is cleaned by alcohol and dried.

3. The silicon wafer of the experimental group is immersed in the platelet PBS solution and is left to stand for 30 minutes at 37° C.

4. The silicon wafer is gently washed by PBS 3 times and put into 2.5% glutaraldehyde for fixation overnight.

5. 50%, 60%, 70%, 80%, 95%, and 100% ethanol are serially added for gradient dehydration, 15 minutes each time.

6. Gold is sprayed, and the platelet aggregation is obtained by the scanning electron microscope.

Experimental Results:

Platelets are a first outpost for hemostasis. After tissue trauma or vascular rupture is detected, platelets will be serially adhered to the collagen fibers exposed at the injury, deformed, and activated, the blood-clotting factors are released and aggregated in a large number to form soft platelet thrombi, and the soft platelet thrombi are then shrunk into compact thrombi. Collagen fiber monomers further surround the soft platelet thrombi to form large thrombi to block the free red blood cells due to subsequent thrombin function, and a further blood outflow from the wound is blocked.

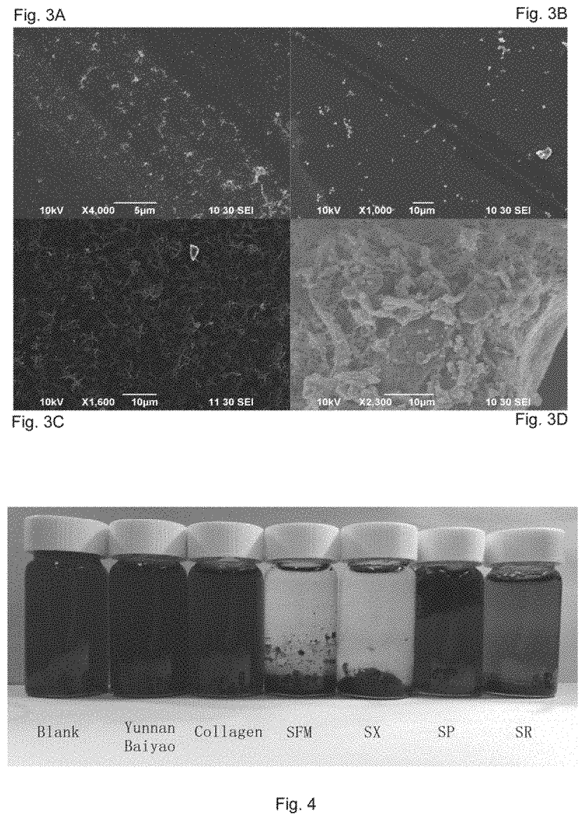

Referring to FIGS. 3A-3D, compared with platelets without undifferentiated deformation scattered and diluted on the blank silicon plate, a large number of the platelets adhere to the silicon plate with the coating of the soluble spongy materials SR and are activated and deformed synchronously, blood-clotting factors are released to enable platelets to begin aggregation reaction and to be stretched to a dendritic structure on a plane of the silicon plate, and a preliminary grid skeleton is formed. In the spongy powders SFM group, it is observed that the platelets are not only adhered to the surfaces of the spongy materials in large quantities but also are highly differentiated, deformed, and aggregated to form the soft platelet thrombi, contracting factors are released, and a firm thrombus layer is finally formed on the surface of the spongy powders SFM. In practical applications, without relying on a formation of fiber protein monomers to provide structural strength, the spongy powders SFM group can save a lot of time spent in the hemostasis process, quickly combine the platelets to provide highly differentiated thrombi, accelerate a hemostasis rate, and avoid a formation difficulty of the thrombi due to massive blood loss and excessively large wounds.

III. Special Hemostatic Property

The most special hemostatic property of the spongy materials is shown in a normal coagulation function after blood with coagulation factor defects is contacted by the spongy materials. In the experiment, platelet-poor blood and fibrinogen removal blood are tested.

The experimental materials are as follows:

Sterile sheep defibrated blood (e.g., sterile sheep fibrinogen removal blood), which is purchased from Nanjing Maojie Microbiology Co., Ltd.

Platelet-poor blood: fresh plasma is taken and centrifuged at 1000 rpm/10 min (1000 revolutions/10 minutes) in a centrifuge to separate the red blood cells and the plasma. The red blood cells are cleaned using repeated centrifugation by PBS solution 3 times, and the plasma is centrifuged again at 3500 rpm/15 min (3500 revolutions/15 minutes). An upper layer, that is, a platelet-poor plasma is taken, the process is repeated 3 times, and the red blood cells and the platelet-poor plasma are mixed according to the original ratio to obtain the platelet-poor blood.

Experimental group: small particles of the spongy powders SFM, the soluble degradable spongy materials SR, large particles of flocculent spongy materials SX, and whole spongy skeletons SP

Control Group: Blank, Collagen, Yunnan Baiyao

Experimental method: the blood-clotting time of whole blood is expressed by a glass test tube method, blood-clotting degree is observed by a pure water lysing free red blood cells method at a macro level, and structures of thrombi are observed by a scanning electron microscope at a micro level.

Experimental Results:

The platelet-poor blood used for the blood-clotting is difficult to clot under normal conditions. After the platelets are activated, the blood-clotting factors are released to activate prothrombin to be converted into the thrombin, so that fibrinogens in the blood surround the platelet thrombi to form fiber protein monomers, and blood clot is finally generated and clotted.

TABLE 3
The blood-clotting time of the platelet-poor blood for all
materials (unit: minutes)

| Groups | 10 mg/mL | 50 mg/mL | 100 mg/mL |
|---|---|---|---|
| Blank | Not clotted | | |
| Collagen | Not clotted | | |
| Yunnan | Not clotted | | |
| Baiyao | | | |
| SFM | 25.49 ± 1.11 | 16.97 ± 1.08 | 13.73 ± 0.46 |
| SX | 40.74 ± 2.91 | 33.52 ± 0.51 | 32.37 ± 0.62 |

| | |
|---|---|
| SP | Not clotted |
| SR | Not completely clotted |

Referring to Table 3 and FIG. 4, it can be seen that the materials in the control groups cannot ultimately promote the platelet-poor blood to be clotted. The spongy materials have a significant hemostatic effect on the platelet-poor blood, and a hemostatic effect of the spongy materials is related to the morphological structure of the spongy materials.

Structures of the spongy powders SFM, the flocculent spongy materials SX, and the soluble degradable spongy materials SP are compared, and the main differences are particle sizes of the spongy materials and material clearances and distances. It is speculated that the whole spongy skeletons SP have mesh structures visible to the naked eye. Spaces between the skeletons are large, so that the red blood cells cannot be firmly adhered and adsorbed. After the skeletons are ground, when particle sizes of the spongy materials are smaller, specific surface areas of the spongy materials are larger, the spaces of the spongy materials are shortened, efficiencies for blocking and adsorbing the red blood cells are higher, and the blood-clotting rate is quicker.

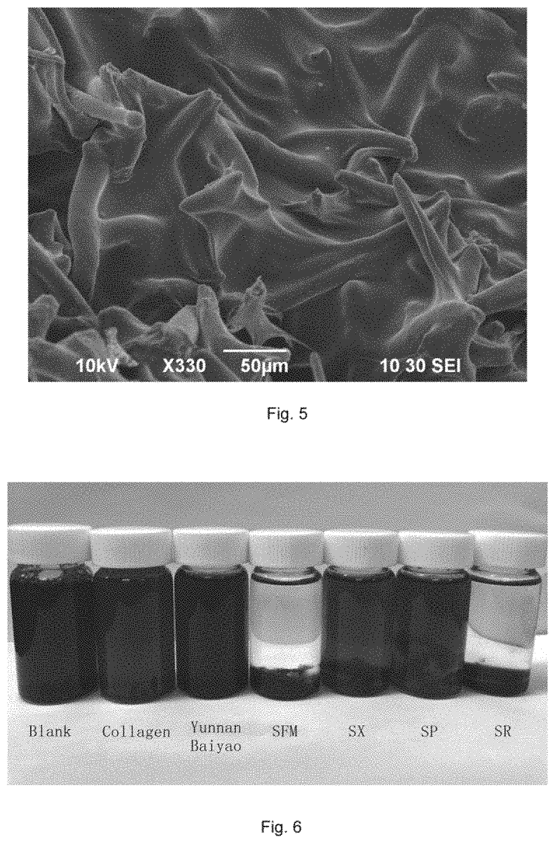

When FIGS. 5 and 6 are compared, it can be seen that surfaces of the thrombi formed by the platelet-poor blood are relatively smooth, and the platelets do not form a support for the thrombi. The soluble spongy materials SR are dissolved in the blood and do not provide stable structures for support. Therefore, it is observed in the experiments that the blood in the soluble spongy materials SR group is not completely clotted. However, the blood forms local blood clots at a bottom due to an interaction of gravity sedimentation and lacking support.

TABLE 4
The blood-clotting time of the sheep fibrinogen removal blood on
all materials (unit: minutes)

| Groups | 10 mg/mL | 20 mg/mL | 40 mg/mL | 60 mg/mL |
|---|---|---|---|---|
| Blank | Not clotted | | | |
| Collagen | Not clotted | | | |
| Yunnan | Not clotted | | | |
| Baiyao | | | | |
| SFM | Not completely | 8.29 ± 0.37 | 3.18 ± 0.21 | 2.1 ± 0.14 |
| | clotted | | | |
| SX | Not clotted | | | |
| SP | Not clotted | | | |
| SR | 4.92 ± 0.21 | 3.71 ± 0.12 | 4.49 ± 0.43 | 9.23 ± 0.48 |

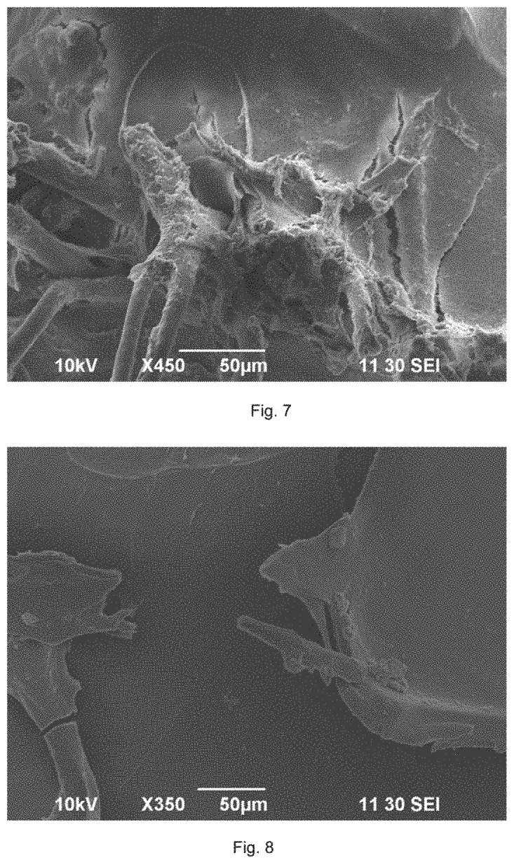

Table 4, FIG. 6, and FIG. 7 illustrate results of the coagulation experiment of the fibrinogen removal blood. As is known, in a formation process of the thrombi, ultimate goals of all blood-clotting mechanisms are to convert the fibrinogen in the blood into a solid fibrin monomer network to cooperate with the platelet thrombi to fix the free red blood cells and plasma components. In the fibrinogen removal blood experiment, the fibrinogens are removed. The experimental results show that only the soluble spongy materials SR and the spongy powders SFM can enable the sheep fibrinogen removal blood to be normally clotted. In combination with the aforementioned experiments, the analysis is as follows.

1. The soluble spongy materials SR are dissolved in the blood and cannot have stable solid structures like other insoluble spongy particles. Compared with the fibrinogen removal test and the platelet-poor blood, it is found that the soluble spongy materials SR group needs the platelets to provide structural assistance to form thrombi during the blood-clotting process, and the blood can be normally clotted without a presence of fiber protein components, so it is speculated that the spongy materials can well replace the fibrinogen in the blood.

2. Compared with the experimental results of the spongy powders SFM, the flocculent spongy materials SX, and the soluble spongy materials SP, it is found that the spongy materials differs from other hemostatic drugs. The other hemostatic drugs, with too high of a concentration, can interfere with blood-clotting reaction, and when a content of the spongy materials in the blood is higher, particle sizes of the spongy materials are smaller, and the blood-clotting performance is better. It is speculated that the spongy materials are not only a simple replacement for fibrinogens, but it is also observed that individual branches of the insoluble spongy powders intersect and are overlapped with each other to form a dense network structure in the micro level, which is similar with a fiber protein net formed by cross-linking with the fiber protein monomers. The spongy materials also have properties of enriching the red blood cells and agglomerating the platelets, thus a coagulation function of the fiber protein is directly replaced, the spongy materials are coagulated with the red blood cells and other coagulation substances in the blood to form the thrombi, a process for producing the fiber protein monomers in the three stages of the blood-clotting process is reduced, and a required blood-clotting time is greatly shortened.

In summary, as a hemostatic material, the spongy materials have good water absorption and biocompatibility. The spongy materials not only have excellent performance in the red blood cells adsorption and the platelets activation, but also can effectively act on special blood with blood-clotting factors disorder. The spongy materials have low cost and high output, which can greatly alleviate problems of hemophilia patients and other special populations, such as expensive drugs and being difficult to afford. The spongy materials have a broad market prospect.

The aforementioned embodiments are merely some embodiments of the present disclosure, and the scope of the disclosure is not limited thereto. Thus, it is intended that the present disclosure cover any modifications and variations of the presently presented embodiments provided they are made without departing from the appended claims and the specification of the present disclosure.

What is claimed is:

1. A method for preparing a hemostatic spongy material or a tissue sealant, comprising the following steps:
(1) taking an adult sponge and washing away fleshy components on a surface of the adult sponge, keeping a skeleton of the adult sponge;
(2) immersing the skeleton of step (1) in a hydrochloric acid (HCl) solution with a concentration of 0.7 to 0.8 mol/L for 2 to 3 days, taking the skeleton out, and washing the skeleton several times with clean water;
(3) immersing the skeleton of step (2) in an NaOH solution with a concentration of 0.1 to 0.2 mol/L for 2 to 3 days, taking the skeleton out, and immersing the skeleton in clean water for 3 to 4 days;
(4) adding the skeleton of step (3) to a Tris-HCl buffer solution, stirring and pulverizing into a homogenous suspension, wherein a concentration of the Tris-HCl buffer solution is 0.1M and a pH is 7.8 at 37° C.; adding 10% trypsin to the homogenous suspension, shaking for enzymolysis for 2 to 3 days to obtain a product;
(5) filtering and separating the product of step (4) to obtain a separated precipitate, immersing and washing the separated precipitate with clean water 2 to 4 times, and drying to obtain spongy precipitate, wherein the spongy precipitate is an insoluble, large branch fiber spongin B;
(6) at least one of:
breaking the spongy precipitate of step (5) into small particles with a freezing grinder, and sieving to obtain spongy powders SFM; or
using a hydrogen peroxide method to degrade the spongy precipitate of step (5) into soluble spongy materials SR.

2. The method according to claim 1, wherein the adult sponge is Dictyoceratida sponge.

3. The method according to claim 1, wherein in step (1), the adult sponge is decayed to decompose an epidermis and the fleshy components, or the adult sponge is put in sea water and vigorously washed to remove the fleshy components on the surface of the adult sponge.

4. The method according to claim 1, wherein in step (2), the HCl solution has a concentration of 0.8 mol/L, and an immersing time is 2 days.

5. The method according to claim 1, wherein in step (3), the NaOH solution has a concentration of 0.1 mol/L.

6. The method according to claim 1, wherein in step (6), a 200-mesh sieve is used to obtain the spongy powders SFM.

7. The method according to claim 1, wherein the hemostatic spongy material has a scattered pore structure.

8. A hemostatic material or a tissue sealant, wherein a main component of the hemostatic material or the tissue sealant or part component of the hemostatic material or the tissue sealant is a sponge grown in sea water or fresh water.

9. The hemostatic material or the tissue sealant according to claim 8, wherein fleshy components on a surface of the sponge are removed.

10. The hemostatic material or the tissue sealant according to claim 8, wherein the sponge is frozen and broken after precipitation to obtain the spongy powders SFM.

11. The hemostatic material or the tissue sealant according to claim 8, wherein a spongy material of the sponge is degraded after precipitation into soluble spongy materials SR.
